SEQUENCE LISTING (35 sequences)

SEQ ID NO: 1 - DNA, 240 bp, Lactococcus lactis
  Constitutive p44 promoter
aacaattgtaacccatacaggagaagggacgatagcaattttttcaataagtagacaaagtagagaataatttaataaaaaactgaaaaaatcacagctaaactcttgttttacttgattttatgttaaaataattaatgagtgtaattgtatataaaattatctgtacacttacctaatttattaaaaaaaaatatgaatcgtgatgtgtgagggaaaggagtcgcttttatggccaaa

SEQ ID NO: 2 - DNA, 195 bp, Artificial Sequence
  Fragment of the promoter p44 corresponding to the nt 1-195 sequence
aacaattgtaacccatacaggagaagggacgatagcaattttttcaataagtagacaaagtagagaataatttaataaaaaactgaaaaaatcacagctaaactcttgttttacttgattttatgttaaaataattaatgagtgtaattgtatataaaattatctgtacacttacctaatttattaaaaaaaaat

SEQ ID NO: 3 - DNA, 62 bp, Artificial Sequence
  Fragment of the promoter p44 corresponding to the nt 88-149 sequence
aaaatcacagctaaactcttgttttacttgattttatgttaaaataattaatgagtgtaatt

SEQ ID NO: 4 - DNA, 72 bp, Artificial Sequence
  Fragment of the promoter p44 corresponding to the nt 88-159 sequence
aaaatcacagctaaactcttgttttacttgattttatgttaaaataattaatgagtgtaattgtatataaaa

SEQ ID NO: 5 - DNA, 123 bp, Artificial Sequence
  Fragment of the promoter p44 corresponding to the nt 27-149 sequence
ggacgatagcaattttttcaataagtagacaaagtagagaataatttaataaaaaactgaaaaaatcacagctaaactcttgttttacttgattttatgttaaaataattaatgagtgtaatt

SEQ ID NO: 6 - DNA, 133 bp, Artificial Sequence
  Fragment of the promoter p44 corresponding to the nt 27-159 sequence
ggacgatagcaattttttcaataagtagacaaagtagagaataatttaataaaaaactgaaaaaatcacagctaaactcttgttttacttgattttatgttaaaataattaatgagtgtaattgtatataaaa

SEQ ID NO: 7 - DNA, 181 bp, Artificial Sequence
  Fragment of the promoter p44 corresponding to the nt 15-195 sequence
atacaggagaagggacgatagcaattttttcaataagtagacaaagtagagaataatttaataaaaaactgaaaaaatcacagctaaactcttgttttacttgattttatgttaaaataattaatgagtgtaattgtatataaaattatctgtacacttacctaatttattaaaaaaaaat

SEQ ID NO: 8 - DNA, 169 bp, Artificial Sequence
  Fragment of the promoter p44 corresponding to the nt 27-195 sequence
ggacgatagcaattttttcaataagtagacaaagtagagaataatttaataaaaaactgaaaaaatcacagctaaactcttgttttacttgattttatgttaaaataattaatgagtgtaattgtatataaaattatctgtacacttacctaatttattaaaaaaaaat

SEQ ID NO: 9 - DNA, 145 bp, Artificial Sequence
  Fragment of the promoter p44 corresponding to the nt 15-159 sequence
atacaggagaagggacgatagcaattttttcaataagtagacaaagtagagaataatttaataaaaaactgaaaaaatcacagctaaactcttgttttacttgattttatgttaaaataattaatgagtgtaattgtatataaaa

SEQ ID NO: 10 - DNA, 165 bp, Artificial Sequence
  PL3 - comprised as a promoter the sequence SEQ ID NO 6, a sequence 5′UTR and the beginning of sequence encoding EXP4 signal sequence
actgactgactgactggacgatagcaattttttcaataagtagacaaagtagagaataatttaataaaaaactgaaaaaatcacagctaaactcttgttttacttgattttatgttaaaataattaatgagtgtaattgtatataaaacggtccgatatatatat

SEQ ID NO: 11 - DNA, 36 bp, Artificial Sequence
  Primer for the amplification of the mature form of NucB protein from Staphylococcus aureus from pLB145 vector
tttaaatttaggatccgcatcacaaacagataacgg

SEQ ID NO: 12 - DNA, 36 bp, Artificial Sequence
  Primer for the amplification of the mature form of NucB protein from Staphylococcus aureus from pLB145 vector
tatatatataggtaccttattgacctgaatcagcgt

SEQ ID NO: 13 - DNA, 897 bp, Artificial Sequence
  Sequence of the DNA Z301 fragment obtained by gene synthesis
agatctttgatcaaggatctgtcagctggttcaactagcggtggtcaaactgttagtaataaaacttattgttttgatgttcggcttaaggatggaaggatttttcaaataaaaaagtaaaaaataatgttaactggttgacattatttttactttgctatataattaaccagtaaactaattatggaggacgaaatactatgagtttagcaaatcaaatcgaccagtttcttggggcaattatgcagtttgcggaaaacaagcatgaaatattactcggcgaatgcgaaagtaatgttaagctaacaagcacgcaagaacatatcttaatgattctagctgcagaggtttcgacaaacgcgagaattgccgagcaactcaagatttcgccagcagcggtaactaaagctctcaaaaaattacaagagcaagaactgattaaatcaagtcgggcaacaaatgacgaacgcgtagtcctttggagcctgacagaaaaagcaattccagttgctaaagaacatgctgctcatcatgagaaaactcttagtacctaccaagaattaggagacaaatttactgacgaagaacaaaaagtgataagtcaattcttatcagtacttactgaagagtttcgatgaagacggtccgtaccttaaggagatataaaaatgaaaaaaataaaccttgctttattaacgctagctactctaatgggagtctcatcaacagccgttgtatttgctgatgatactggcaggtttggatccatgggacatatgctcgagctagcaggcctgcagtcgacgcggccgcggtaccaagcttctagaattcgcggccgcctgcaggtcgacggtatcgatagcccgcctaatgagcgggcttttttttgatatc

SEQ ID NO: 14 - DNA, 93 bp, Artificial Sequence
  Modified nucleotidic sequence by directed mutagenesis of the pGTP FZR 303 vector
ccgttgtattcgctgagaccggatccatgggacatatgctcgagctagcaggcctgcagtcgacgcggccgcggtaccaagcttctagaattc

SEQ ID NO: 15 - DNA, 756 bp, Artificial Sequence
  Optimized nucleotidic sequence encoding the Apo-AI protein
ggtctcttcgctgatgaaccaccacaatcaccatgggatcgtgttaaagatcttgctacagtttatgttgatgttcttaaagattcaggtcgtgattatgtttcacaatttgaaggttcagctcttggtaaacaacttaatcttaaacttcttgataattgggattcagttacatcaacattttcaaaacttcgtgaacaacttggtccagttacacaagaattttgggataatcttgaaaaagaaacagaaggtttgcgtcaagaaatgtctaaagatcttgaagaagttaaagcaaaagttcaaccatatcttgatgattttcaaaaaaaatggcaagaagaaatggaactttatcgtcaaaaagttgaaccacttcgtgctgaattacaagaaggtgctcgtcaaaaacttcatgaacttcaagaaaaactttcaccacttggtgaagaaatgcgtgatcgtgctcgtgctcatgttgatgctcttcgtacacatcttgctccatattcagatgaacttcgtcaacgtttagctgctcgtcttgaagctcttaaagaaaatggtggtgctcgtcttgctgaatatcatgctaaagctacagaacatctttcaacactttcagaaaaagctaaaccagctcttgaagatttacgtcaaggtcttttgccagttcttgaatcatttaaagtttcatttctttcagctttggaagaatatacaaaaaaacttaatacacaataataataatctaga

SEQ ID NO: 16 - DNA, 210 bp, Artificial Sequence
  PL1 - derived sequence from P10 promoter
actgactgactgactacaaaaacctaatttattttaaatttttgcatgtaatgagtttattcttgacaacttttgggaaacttggtatactaatatagtcgtttaagagaaacgcaggcgtggctcaactggatagagtacctgactacgaatcaggcggttgtaggttcgaatcctaccgcttgcataaataacggtccgatattatat

SEQ ID NO: 17 - DNA, 176 bp, Artificial Sequence
  PL2 - derived sequence from P21 promoter
actgactgactgactcaaatcccacactctatcgtcaaacaatttcaaaatttatgaagacttataatccgtaatttattgacttttatatcagtgatttatgagttttttcttgacagaagaaggcgaaaaatggtattatatttaggtactgttttgcggtccgatatatatat

SEQ ID NO: 18 - DNA, 105 bp, Artificial Sequence
  PL4 - derived sequence from PUSP45 promoter
actgactgactgactgtgttttgtaatcataaagaaatattaaggtggggtaggaatagtataatatgtttattcaaccgaacttaatcggtccgatatatatat

SEQ ID NO: 19 - DNA, 229 bp, Artificial Sequence
  PL3
agatctggacgatagcaattttttcaataagtagacaaagtagagaataatttaataaaaaactgaaaaaatcacagctaaactcttgttttacttgattttatgttaaaataattaatgagtgtaattgtatataaaattgctatataattaaccagtaaactaattaattaataccttaaggagatataaaaatgaaaaaaataaaccttgctttattaacgctagc

SEQ ID NO: 20 - DNA, 315 bp, Artificial Sequence
  Sequence comprising the sequence P44
agatctttgatctaacaattgtaacccatacaggagaagggacgatagcaattttttcaataagtagacaaagtagagaataatttaataaaaaactgaaaaaatcacagctaaactcttgttttacttgattttatgttaaaataattaatgagtgtaattgtatataaaattatctgtacacttacctaatttattaaaaaaaaatatgaatcgtgatgtgtgagggaaaggagtcgcttttatggccaaacggtccgtaccttaaggagatataaaaatgaaaaaaataaaccttgctttattaacgctagc

SEQ ID NO: 21 - DNA, 223 bp, Artificial Sequence
  PL3A - from truncated p44 promoter
agatctaacaattgtaacccatacaggagaagggacgatagcaattttttcaataagtagacaaagtagagaataatttaataaaaaactgaaaaaatcacagctaaactcttgttttacttgattttatgttaaaataattaatgagtgtaattgtatataaaattatctgtacacttacctaatttattaaaaaaaaatatgaatcgtgatgtgtgaggga

SEQ ID NO: 22 - DNA, 201 bp, Artificial Sequence
  PL3B - from truncated p44 promoter
agatctaacaattgtaacccatacaggagaagggacgatagcaattttttcaataagtagacaaagtagagaataatttaataaaaaactgaaaaaatcacagctaaactcttgttttacttgattttatgttaaaataattaatgagtgtaattgtatataaaattatctgtacacttacctaatttattaaaaaaaaat

SEQ ID NO: 23 - DNA, 220 bp, Artificial Sequence
  PL3C - from truncated p44 promoter
agatctggacgatagcaattttttcaataagtagacaaagtagagaataatttaataaaaaactgaaaaaatcacagctaaactcttgttttacttgattttatgttaaaataattaatgagtgtaattgtatataaaattatctgtacacttacctaatttattaaaaaaaaatatgaatcgtgatgtgtgagggaaaggagtcgcttttatggccaaa

SEQ ID NO: 24 - DNA, 232 bp, Artificial Sequence
  PL3D - from truncated p44 promoter
agatctatacaggagaagggacgatagcaattttttcaataagtagacaaagtagagaataatttaataaaaaactgaaaaaatcacagctaaactcttgttttacttgattttatgttaaaataattaatgagtgtaattgtatataaaattatctgtacacttacctaatttattaaaaaaaaatatgaatcgtgatgtgtgagggaaaggagtcgcttttatggccaaa

SEQ ID NO: 25 - DNA, 155 bp, Artificial Sequence
  PL3E - from truncated p44 promoter
agatctaacaattgtaacccatacaggagaagggacgatagcaattttttcaataagtagacaaagtagagaataatttaataaaaaactgaaaaaatcacagctaaactcttgttttacttgattttatgttaaaataattaatgagtgtaatt

SEQ ID NO: 26 - DNA, 237 bp, Artificial Sequence
  PL3F - from truncated p44 promoter
agatctaacaattgtaacccatacaggagaagggacgatagcaattttttcaataagtagacaaagtagagaataatttaataaaaaactgaaaaaatcacagctaaactcttgttttacttgattttatgttaaaataattaatgagtgtaattgtatataaaattatctgtacacttacctaatttattaaaaaaaaatatgaatcgtgatgtgtgagggaaaggagtcgctttt

SEQ ID NO: 27 - DNA, 185 bp, Artificial Sequence
  PL3G - from truncated p44 promoter
agatctaacaattgtaacccatacaggagaagaaaatcacagctaaactcttgttttacttgattttatgttaaaataattaatgagtgtaattgtatataaaattatctgtacacttacctaatttattaaaaaaaaatatgaatcgtgatgtgtgagggaaaggagtcgcttttatggccaaa

SEQ ID NO: 28 - DNA, 94 bp, Artificial Sequence
  PL3H - from truncated p44 promoter
agatctaacaattgtaacccatacaggagaagaaaatcacagctaaactcttgttttacttgattttatgttaaaataattaatgagtgtaatt

SEQ ID NO: 29 - DNA, 246 bp, Artificial Sequence
  Sequence comprising the sequence P44
agatctaacaattgtaacccatacaggagaagggacgatagcaattttttcaataagtagacaaagtagagaataatttaataaaaaactgaaaaaatcacagctaaactcttgttttacttgattttatgttaaaataattaatgagtgtaattgtatataaaattatctgtacacttacctaatttattaaaaaaaaatatgaatcgtgatgtgtgagggaaaggagtcgcttttatggccaaa

SEQ ID NO: 30 - DNA, 280 bp, Artificial Sequence
  PL3B1  - from truncated p44 promoter
agatctaacaattgtaacccatacaggagaagggacgatagcaattttttcaataagtagacaaagtagagaataatttaataaaaaactgaaaaaatcacagctaaactcttgttttacttgattttatgttaaaataattaatgagtgtaattgtatataaaattatctgtacacttacctaatttattaaaaaaaaatatgaatcgtgatgtgtgcggtccgtaccttaaggagatataaaaatgaaaaaaataaaccttgctttattaacgctagc

SEQ ID NO: 31 - DNA, 274 bp, Artificial Sequence
  PL3B2 - from truncated p44 promoter
agatctaacaattgtaacccatacaggagaagggacgatagcaattttttcaataagtagacaaagtagagaataatttaataaaaaactgaaaaaatcacagctaaactcttgttttacttgattttatgttaaaataattaatgagtgtaattgtatataaaattatctgtacacttacctaatttattaaaaaaaaatatgaatcgtgacggtccgtaccttaaggagatataaaaatgaaaaaaataaaccttgctttattaacgctagc

SEQ ID NO: 32 - DNA, 268 bp, Artificial Sequence
  PL3B3 - from truncated p44 promoter
agatctaacaattgtaacccatacaggagaagggacgatagcaattttttcaataagtagacaaagtagagaataatttaataaaaaactgaaaaaatcacagctaaactcttgttttacttgattttatgttaaaataattaatgagtgtaattgtatataaaattatctgtacacttacctaatttattaaaaaaaaatatgaacggtccgtaccttaaggagatataaaaatgaaaaaaataaaccttgctttattaacgctagc

SEQ ID NO: 33 - DNA, 218 bp, Artificial Sequence
  PL3B1 - from truncated p44 promoter nt 1-212
agatctaacaattgtaacccatacaggagaagggacgatagcaattttttcaataagtagacaaagtagagaataatttaataaaaaactgaaaaaatcacagctaaactcttgttttacttgattttatgttaaaataattaatgagtgtaattgtatataaaattatctgtacacttacctaatttattaaaaaaaaatatgaatcgtgatgtgtg

SEQ ID NO: 34 - DNA, 204 bp, Artificial Sequence
  PL3B3 - from truncated p44 promoter nt  1-198
agatctaacaattgtaacccatacaggagaagggacgatagcaattttttcaataagtagacaaagtagagaataatttaataaaaaactgaaaaaatcacagctaaactcttgttttacttgattttatgttaaaataattaatgagtgtaattgtatataaaattatctgtacacttacctaatttattaaaaaaaaatatg

SEQ ID NO: 35 - DNA, 211 bp, Artificial Sequence
  PL3B2  - from truncated p44 promoter nt 1-205
agatctaacaattgtaacccatacaggagaagggacgatagcaattttttcaataagtagacaaagtagagaataatttaataaaaaactgaaaaaatcacagctaaactcttgttttacttgattttatgttaaaataattaatgagtgtaattgtatataaaattatctgtacacttacctaatttattaaaaaaaaatatgaatcgtg

(19) United States
(12) Patent Application Publication
(10) Pub. No.: US 2011/0020866 A1
(43) Pub. Date: Jan. 27, 2011

(54) Promoter sequence for the expression of recombinant proteins in Lactococcus lactis

(76) Inventors: Hervé Ginisty, Ramonville Saint Agne (FR); Eric Devic, Saint Orens de Gameville (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/805,050

(22) Filed: Jul. 8, 2010

(30) Foreign Application Priority Data
Jul. 9, 2009 (FR) 0954785

(51) Int. Cl. C12P 21/02; C07H 21/04; C12N 15/63; C12N 15/74; C12N 1/21

(52) U.S. Cl. 435/69.1; 536/23.7; 435/320.1; 435/252.3

(57) ABSTRACT

The present invention relates to new DNA sequences that function as promoters, expression vectors containing such sequences, and host cells transformed with these vectors, in particular lactic acid bacteria such as Lactococcus lactis. The invention also relates to the use of these promoters for the production of heterologous proteins, in particular therapeutic or vaccine-related proteins.

The present invention relates to novel DNA sequences that function as promoters, expression vectors containing such sequences, and host cells transformed with these vectors, in particular lactic acid bacteria such as Lactococcus lactis. The invention also describes use of these promoters for the production of heterologous proteins, in particular therapeutic or vaccine-related proteins.

Recent progress in the area of molecular biology has enabled microorganisms to be modified to make them produce heterologous proteins of interest. In particular, many genetic studies have focused on cells from mammals or birds, or prokaryotic cells such as bacteria, in particular E. coli. However, given the special features of these cells (the possibility of obtaining glycosylated or non-glycosylated proteins, presence of an oncogenic virus, limited yield, etc.), the industrial application of these new production methods is still limited, particularly by problems relating to the efficacy or safety of gene expression in these recombinant microorganisms.

To increase the performance of these production methods, studies have been conducted on a regular basis in order in particular to isolate strong novel constitutive promoters or to improve the culture conditions of these microorganisms or the secretion and/or extraction systems for these proteins.

Lactic acid bacteria are currently used in the agri-food industry. Aside from this historical application, these bacteria are of increasing interest as hosts for the production of heterologous proteins and are increasingly used for this purpose. The heterologous proteins expressed in lactic acid bacteria may be very different in nature and application. However, the harmlessness of these bacterial strains makes them particularly useful for the production of recombinant proteins with therapeutic or vaccine-related goals. The ability of bacterial strains to directly secrete proteins of interest into the culture supernatant grants them an additional advantage by enabling easy purification of the protein of interest for bio-production applications or permitting use of the bacterial strain as a delivery vector for the protein of interest in situ in the patient.

The expression of recombinant proteins in lactic acid bacteria requires the use of expression tools enabling optimization of the production of the protein of interest. The promoter is a key element of these expression tools, playing a crucial role in the level of expression of the protein of interest. In general, it is often thought that the stronger the promoter, the more optimal the potential expression of the protein of interest. However, although a high transcription level is necessary to obtain optimal expression of the protein, a transcription level that is too high may have deleterious effects on the expression level of the protein of interest in actual production. Several different causes may be responsible for these sub-optimal levels of expression. The “energetic” overhead associated with a very high level of expression of the protein confers a selective disadvantage on the strains expressing the protein of interest by reducing their growth rates. Instability of the strain may result from this selective disadvantage, with the risk of spontaneous mutants being selected that have lost the capacity to express the protein of interest. In cases where proteins are secreted, control of the intracellular level of expression is even more crucial. In fact, over-expression of the protein of interest requires that it be exported through the plasma membrane of the bacterium. This post-transcription stage may become limiting and lead to the intracellular accumulation of the immature form of the protein of interest and the potential induction of stress-resistance response mechanisms (Hyyrylainen et al., 2002).

Control of the right transcription level and therefore the choice of an optimal promoter constitute essential elements of recombinant protein expression. Although lactic acid bacteria have been studied for many years and the genome sequences of some are known, currently only a small number of promoters are used in the expression of heterologous proteins. These promoters are divided into two types: inducible promoters and constitutive promoters.

Inducible promoters permitting expression of the protein of interest to be triggered or stopped are of interest as proteins that are toxic or interfere with the metabolism of the host bacterium. Several types of inducible promoters have been described in L. lactis: the promoter nisin (PnisA) (de Vos et al., 1995; Kuipers et al., 1995), a thermo-inducible promoter (Nauta, 1997), an promoter that can be induced by phage φ31 (Walker, 1998), the promoter p170 (Madsen et al., 1999), the promoter xylose (P_{Xyl}) (Miyoshi et al., 2004) and the promoter zinc (P_{Zn}) (Llull et al., 2004).

Constitutive promoters permitting continuous expression of the protein have the advantage of enabling the protein to be expressed throughout the culture and do not require an induction phase. However, since the transcription level of these promoters cannot be modulated, choice of a promoter with an optimal transcription level is essential. Several constitutive promoters of various strengths have been described in L. lactis (Koivula et al., 1991; van der Vossen et al., 1987; and van Asseldonk et al., 1990). Several of these promoters have been used for the expression of heterologous proteins (for review: Morello 2008, de Vos 1999).

The inventors demonstrated that it was possible to produce very clearly improved levels of expression of recombinant proteins, in particular secreted proteins, in Lactococcus lactis when these proteins are expressed under the control of new promoter sequences derived from the constitutive promoter P44 with the sequence SEQ ID NO: 1, derived from Lactococcus lactis.

Thus, the subject matter of the present invention is an isolated nucleic acid characterized in that it is a fragment of the SEQ ID NO: 1 sequence, said fragment being characterized in that is chosen from:
a) a fragment contained in the nucleotides 1 to 127 (nt 1-127) sequence (PL3A of sequence SEQ ID NO: 21), terminals included, of the SEQ ID NO: 1 sequence, with this fragment containing at least the nt 88-149 sequence (SEQ ID NO: 3) of the SEQ ID NO: 1 sequence, preferably containing at least the nt 88-159 sequence (SEQ ID NO: 4), at least the nt 27-149 sequence (SEQ ID NO: 5) or at least the nt 27-159 sequence (SEQ ID NO: 6) of the SEQ. ID NO: 1 sequence; or preferably
b) a fragment contained in the sequence nt 1-215, nt 1-214, nt 1-213, nt 1-212, nt 1-211, nt 1-210, nt 1-209, nt 1-208, nt 1-207, nt 1-206, nt 1-205, nt 1-204, nt 1-203, nt 1-202, nt 1-201, nt 1-200, nt 1-199, nt 1-198, nt 1-197, nt 1-196 or nt 1-195 of the SEQ ID NO: 1 sequence, with this fragment containing at least the nt 88-149 sequence (SEQ ID NO: 3) of the sequence SEQ ID NO: 1, preferably containing at least the nt 88-159 sequence (SEQ ID NO: 4), preferably containing at least the nt 27-149 sequence (SEQ ID NO: 5) or at least the nt 27-159 sequence (SEQ ID NO: 6) of the SEQ ID NO: 1 sequence; or
c) a fragment whose sequence is at least 90%, and preferably at least 95%, 97%, 98% and 99%, identical to the sequence of the fragment as defined in a) or b).

Preferably, the subject matter of the present invention is an isolated nucleic acid, characterized in that it is:
a) a fragment of the SEQ ID NO: 1 sequence, said fragment being characterized in that it is chosen from the fragments contained in the SEQ ID NO: 2 sequence, terminals included, and corresponding to the nt 1-195 sequence of the SEQ ID NO: 1 sequence, with this fragment containing at least the nt 88-149 sequence (SEQ ID NO: 3) of the SEQ ID NO: 1 sequence, or
b) a fragment whose sequence is at least 90%, 95%, 97%, 98% and 99% identical to the sequence of the fragment as defined in a).

The SEQ ID NO: 1 sequence corresponds here to promoter P44 (or p44) with the following sequence:

AACAATTGTAACCCATACAGGAGAAGGGACGATAGCAATTTTTT
CAATAAGTAGACAAAGTAGAGAATAATTTAATAAAAAACTGAAA
AAATCACAGCTAAACTCTTGTTTTACTTGATTTTATGTTAAAAT
AATTAATGAGTGTAATTGTATATAAAATTATCTGTACACTTAAT
TTATTAAAAAAAAATATGAATCGTGATGTGAGGGAAAGGAGTCG
CTTTATGGCCAAA.

The percentage of identity between two nucleic acid sequences designates the degree of identity between the two nucleic acid sequences along their entire length. If the sequences under consideration are of different lengths, the % identity is expressed as a function of the total length of the shortest sequence. To calculate the % identity, the two sequences are superimposed so as to maximize the number of identical bases, with gaps allowed. The number of identical bases is then divided by the total number of bases with the shortest sequence.

The percentage of identity between two sequences can also be defined using the “BLAST 2 Sequences” program. This program uses the BLAST algorithm for comparing DNA-DNA sequences two by two. A version of this program is available on the website http://www.ncbi.nlm.nih.gov/blast/bl2seq/bl2.html

In a more preferable embodiment of the invention, the subject matter of the invention is a nucleic acid according to the invention, characterized in that said fragment is chosen from:
a) a fragment contained in the nt 1-195 sequence (SEQ ID No: 2), terminals included, of the SEQ ID No: 1 sequence, with this fragment containing at least the nt 88-159 sequence (SEQ ID NO: 4) of the ID NO: 1 sequence, or
b) a fragment whose sequence is at least 90%, and preferably at least 95%, 97%, 98% and 99%, identical to the sequence of the fragment as defined in a).

In a more preferable embodiment of the invention, it relates to a nucleic acid according to the invention, characterized in that said fragment is chosen from:
a) a fragment contained in the nt 1-195 sequence (SEQ ID NO: 2), terminals included, of the SEQ ID NO: 1 sequence and containing at least the nt 27-149 sequence (SEQ ID NO: 5) of the SEQ ID NO: 1 sequence; or
b) a fragment whose sequence is at least 90%, and preferably at least 95%, 97%, 98% and 99%, identical to the sequence of the fragment as defined in a).

Even more preferably, the present invention relates to a nucleic acid according to the invention, characterized in that said fragment is chosen from:
a) a fragment contained in the nt 1-195 sequence (SEQ ID NO: 2), terminals included, of the SEQ ID NO: 1 sequence and containing at least the nt 27-159 sequence (SEQ ID NO: 6) of the SEQ ID NO: 1 sequence; or
b) fragment whose sequence is at least 90%, and preferably at least 95%, 97%, 98% and 99%, identical to the sequence of the fragment as defined in a).

The invention also relates to an isolated nucleic acid characterized in that it is:
a) a fragment of the SEQ ID NO: 1 sequence, said fragment being characterized in that it is chosen from the fragments contained in the nt 15-195 sequence (SEQ ID NO: 7) terminals included, with this fragment comprising at least one sequence chosen from the following sequence groups:
   nt 88-149 sequence (SEQ ID NO: 3) of the SEQ ID NO: 1 sequence,
   nt 88-159 sequence (SEQ ID NO: 4) of the SEQ ID NO: 1 sequence, and
   nt 27-159 sequence (SEQ ID NO: 6) of the SEQ ID NO: 1 sequence; or
b) a fragment whose sequence is at least 90%, and preferably at least 95%, 97%, 98% and 99%, identical to the sequence of the fragment as defined in a).

The invention also relates to an isolated nucleic acid characterized in that it is:
a) a fragment of the SEQ ID NO: 1 sequence, said fragment being characterized in that it is chosen from the fragments contained in the nt 27-195 sequence (SEQ ID NO: 8) terminals included, with this fragment comprising at least one sequence chosen from the following sequence groups:
   nt 88-149 sequence (SEQ ID NO: 3) of the SEQ ID NO: 1 sequence,
   nt 88-159 sequence (SEQ ID NO: 4) of the SEQ ID NO: 1 sequence, and
   nt 27-159 sequence (SEQ ID NO: 6) of the SEQ ID NO: 1 sequence; or
b) a fragment whose sequence is at least 90%, and preferably at least 95%, 97%, 98% and 99%, identical to the sequence of the fragment as defined in a).

The invention also contains an isolated nucleic acid characterized in that it is:
a) a fragment of the SEQ ID NO: 1 sequence, said fragment being characterized in that it is chosen from among the fragments contained in the nt 15-159 sequence (SEQ ID NO: 9), terminals included, with this fragment containing at least one sequence chosen from among the following sequence groups:
   nt 88-149 sequence (SEQ ID NO: 3) of the SEQ ID NO: 1 sequence,
   nt 88-159 sequence (SEQ ID NO: 4) of the SEQ ID NO: 1 sequence, and
   nt 27-159 sequence (SEQ ID NO: 6) of the SEQ ID NO: 1 sequence; or
b) a fragment whose sequence is at least 90%, and preferably at least 95%, 97%, 98% and 99%, identical to the sequence of the fragment as defined in a).

In an even more preferable embodiment of the invention, it relates to an isolated nucleic acid characterized in that it is a fragment of the SEQ ID NO: 1 sequence chosen from among the fragments of SEQ ID NO: 2 to SEQ ID NO: 9 sequences or the fragments whose sequence is at least 90% identical, and preferably at least 95%, 97%, 98% and 99%, to one of the SEQ ID NO: 2 to SEQ ID NO: 9 sequences.

Likewise, in an even more preferable embodiment of the invention, it relates to an isolated nucleic acid characterized in that it the fragment having the nt 27-159 sequence (SEQ ID NO: 6) or a fragment whose sequence is at least 90%, and preferably at least 95%, 97%, 98% and 99%, of the sequence of the SEQ ID NO: 6 sequence fragment.

According to the invention, said nucleic acids of the invention have promoter activity.

The invention's promoter activity for nucleic acid transcription may be verified for example by introducing recombinant DNA containing a reporter gene such as luciferase, under control of the promoter sequence studied, into the host cell under consideration; the expression of this gene may then be demonstrated in the cellular host under consideration.

Thus, the invention relates to a promoter consisting of a nucleic acid according to the present invention or containing a nucleic acid according to the present invention as a promoter sequence.

In one particular embodiment, the invention relates to a nucleic acid termed PL3 of sequence SEQ ID NO: 10, containing as a promoter the SEQ ID NO: 6 sequence, a 5′UTR sequence and the beginning of the sequence coding for the EXP4 signal sequence, with the following sequence:

PL3:
ACTGACTGACTGACTGGACGATAGCAATTTTTTCAATAAGTAGACAAA
GTAGAGAATAATTTAATAAAAAACTGAAAAAATCACAGCTAAACTCTT
GTTTTACTTGATTTTATGTTAAAATAATTAATGAGTGTAATTGTATAT
AAAACGGTCCGATATATATAT.

In another embodiment of the invention, the invention relates to an expression vector containing (as a promoter) a promoter comprised of a nucleic acid according to the present invention or containing (as a promoter sequence) a nucleic acid according to the present invention.

Preferably, this expression vector carries a gene of interest or contains a nucleic acid of interest coding for a protein of interest, preferably heterologous to the cellular host transformed by this expression vector, characterized in that this gene of interest or said nucleic acid is under the control of a promoter or a nucleic acid according to the invention.

Of these expression vectors, those that also contain the means necessary for expression of said gene of interest, its replication and, if applicable, selection of the transformed cells are considered preferable.

Of these expression vectors, the PGTP_FZ301 or PGTP_FZR301 vectors are preferred as described in the following examples, or any expression vector adapted for Lactococcus lactis.

Preferably, said gene of interest codes for a protein that is therapeutic, vaccine-associated, diagnostic, cosmetic or agri-food related.

Of these proteins, the following may be cited as a non-exhaustive list:
therapeutic antibodies or their fragments, in particular anti-cancer antibodies; proteins derived from blood products such as serum albumin, alpha or beta-globulin, factor VIII, factor IX, Von Willebrand's factor, fibronectin, alpha-1 antitrypsin, etc.; hormones such as insulin and its variants, glucagon, lymphokines such as interleukins, interferons, colony-stimulating factors (G-CSF, CM-CSF, M-CSF, etc.), TNF, TRF, etc.; growth factors such as growth hormone, erythropoietin, FGF, PEGF, PDGF, TGF, etc.;
antigens capable of inducing effective and protective immune reactions in mammals, particularly in humans, and especially for the preparation of anti-infective vaccines (HIV, HCV, HBS, Mycobacterium tuberculosis, SRV, HSV, influenza virus, vaccinia virus, Chlamydia pneumoniae or trachomatis, etc.);
enzymes such as amylases, lipases, proteases, chymosin, superoxide dismutase, catalase, etc.); or
fusion proteins.

Another embodiment of the invention consists of host cells, characterized in that they are transformed by an expression vector according to the invention, preferably chosen from bacteria, and more preferably from the genus Lactococcus, more particularly Lactococcus lactis.

The transformed cells of the invention may be obtained by any method allowing foreign DNA to be introduced into a cell. This may comprise transformation, electroporation, conjugation, fusion or any other method known to those skilled in the art.

As regards the transformation: various protocols have been described in the prior art. In particular it may be accomplished by treating entire cells in the presence of polyethylene glycol or ethylene glycol or dimethyl sulfoxide (DMSO). The method termed electroporation is well known by those skilled in the art of electroporation.

The methods classically used in molecular biology are well known to those skilled in the art and are fully described in the literature (see in particular reference works such as Maniatis T. et al., “Molecular Cloning, a Laboratory Manual”, Cold Spring Harbor Laboratory, Cold Spring Harbor, N.Y., latest edition).

In a final embodiment, the invention relates to a procedure for the production of a heterologous protein containing the culture of a host cell according to the invention.

In a preferred embodiment, the invention relates to a procedure for the production of a heterologous protein in a cell, characterized in that it contains:
a) the culture of a host cell according to the invention transformed by a vector or nucleic acid containing a gene of interest coding for said heterologous protein, under conditions permitting stable expression of said protein; and
b) extraction of the heterologous protein from the cells or culture supernatant.

Preferably, the heterologous protein is a secreted protein.

In the event that the protein coded by the exogenous gene is secreted naturally, the sequence coding for this protein is preferably preceded by a signal sequence. This signal sequence, chosen based on the host cell, has the role of allowing the recombinant protein to be exported outside the cytoplasm, enabling the recombinant protein to take a conformation close to that of the natural protein and facilitates its purification considerably. This signal sequence may be cleaved, either in a single phase by a signal peptidase which releases the mature protein, with the eliminated sequence usually being called pre-sequence or signal peptide sequence, or in several stages when this signal sequence includes (in addition to the sequence eliminated by the signal peptidase or pre-sequence) a sequence eliminated later during one or several proteolytic events, called a pro-sequence.

Others advantages of the present invention will appear upon reading the following examples, which should be considered as illustrative but in no way exhaustive.

### FIGURE LEGENDS

FIG. 1: PLB145 vector map: repA and repC are plasmid replication genes; ssi and on designate respectively the region containing the single strand initiation signals and the replication origin. Cm designates the gene for resistance to chloramphenicol. pZn designates the promoter zinc; zitR the gene coding for the repressor protein of the zinc promoter; and SPEXP4:NucB the fusion protein between the signal peptide EXP4 and nuclease B; ter designates the transcription terminator.

FIG. 2A: pGTP FZ 301 vector map: repA and repC are plasmid replication genes; ssi and on designate respectively the region containing the single strand initiation signals and the replication origin. Cm designates the gene for resistance to chloramphenicol. pZn designates the promoter zinc; zitR the gene coding for the repressor protein of the zinc promoter and PSexp4 the signal peptide EXP4; ter designates the transcription terminators.

FIG. 2B: Nucleotide sequence of the DNA Z301 fragment obtained from gene synthesis.

FIG. 3A: pGTP FZR 303 vector map: repA and repC are plasmid replication genes; ssi and on designate respectively the region containing the single strand initiation signals and the replication origin. Cm designates the gene for resistance to chloramphenicol. pZn designates the promoter zinc; zitR the gene coding for the repressor protein of the zinc promoter and PSExp4 the signal peptide EXP4. ter designates the transcription terminator.

FIG. 3B: Nucleotide sequence modified by directed mutagenesis (region 729 to 822) Restriction sites BamHI and Eco RI are indicated in bold.

FIG. 4: Optimized nucleotide sequence coding for the Apo-AI protein (obtained by gene synthesis).

FIG. 5A: Table describing the origin sequences from which the 4 promoter sequences PL1, PL2, PL3 and PL4 were designed.

FIG. 5B: Nucleotide sequences of 4 synthetic DNA fragments generated for cloning promoter sequences PL1, PL2, PL3 and PL4. The region corresponding to the restriction site RsrII is indicated in bold.

FIG. 6A: Nucleotide sequence of synthesized DNA fragments corresponding to promoter regions p44 and PL3. The restriction sites BglII and NheI are indicated in bold. The regions corresponding to the sequence coding for the signal peptide EXP4 is indicated in small letters.

FIG. 6B: Alignment of synthetic DNA sequence of the fragment PL3A, PL3B, PL3C, PL3D, PL3E, PL3F, PL3G and PL3H illustrating different deletions of p44 promoter, with these sequences containing the restriction site “AGATCT” in 5′.

FIG. 7: Level of expression of the Apo-AI protein in the culture supernatant obtained following 8 hours of culturing based on different promoter sequences used PL1, PL2, PL3 or PL4.

FIG. 8A: Level of expression of the Apo-AI protein in the culture supernatant obtained following 8 hours of culturing based on different promoter sequences used: PL3 or p44.

FIG. 8B: Level of expression of the NucB protein in the culture supernatant obtained following 8 hours of culturing based on different promoter sequences used: PL3 or p44.

FIG. 9A: Level of expression of the protein NucB in the culture supernatant obtained following 8 hours of culturing based on the promoter sequence used: P44, PL3, PL3A, PL3B, PL3C, PL3D, PL3E, PL3F, PL3G, PL3H. The level of expression of NucB is expressed in relative values compared to that obtained with the P44 promoter (also called p44).

FIG. 9B: Schematic representation of different deletions made in the PL3A to PL3H promoters; the portion corresponding to the promoter region is presented by a red dash; the regions corresponding to the restriction sites used for cloning and to 5′UTR specific to the signal peptide EXP4 are represented by a blue dash. The blue rectangle annotated “EXP4” give a diagram of the codons coding for the first amino acids of the EXP4 signal peptide. The enzyme restriction sites BglII and NheI and the position of the ATG initiation codon are indicated. The regions corresponding to deletions are illustrated by dotted lines.

FIG. 10A: Schematic representation of different deletions made in the PL3B1, PL3B2 and PLB3 promoters (PL3A and PL3B promoters are indicated as references); the portion corresponding to the promoter region is presented by a red dash; the regions corresponding to the restriction sites used for cloning and to 5′UTR specific to the signal peptide EXP4 are represented by a blue dash. The blue rectangle annotated “EXP4” give a diagram of the codons coding for the first amino acids of the EXP4 signal peptide. The enzyme restriction sites BglII and NheI and the position of the ATG initiation codon are indicated. The regions corresponding to deletions are illustrated by dotted lines.

FIG. 10B: Nucleotide sequences of 3 synthetic DNA fragments generated for cloning promoter sequences PL3B1, PL3B2 and PL3B3. The region corresponding to the restriction sites BglII and NheI are indicated in bold.

### EXAMPLE 1

### Materials and Methods

### A) Expression Vectors

### PGTP_FZ301

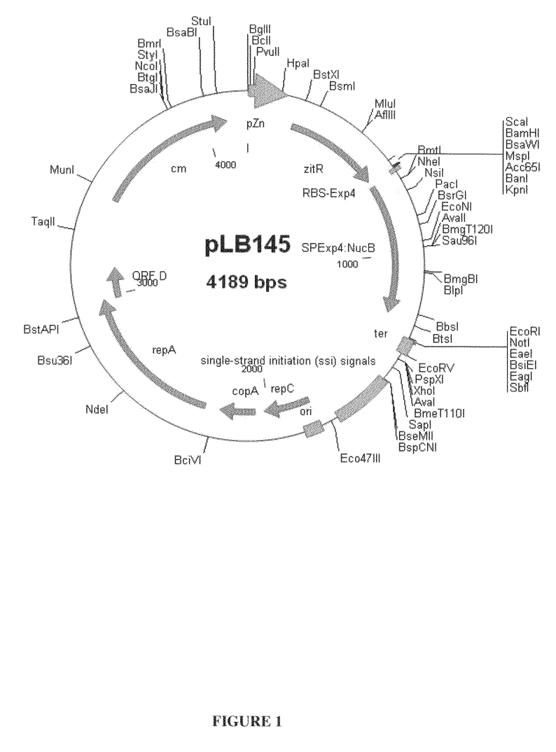

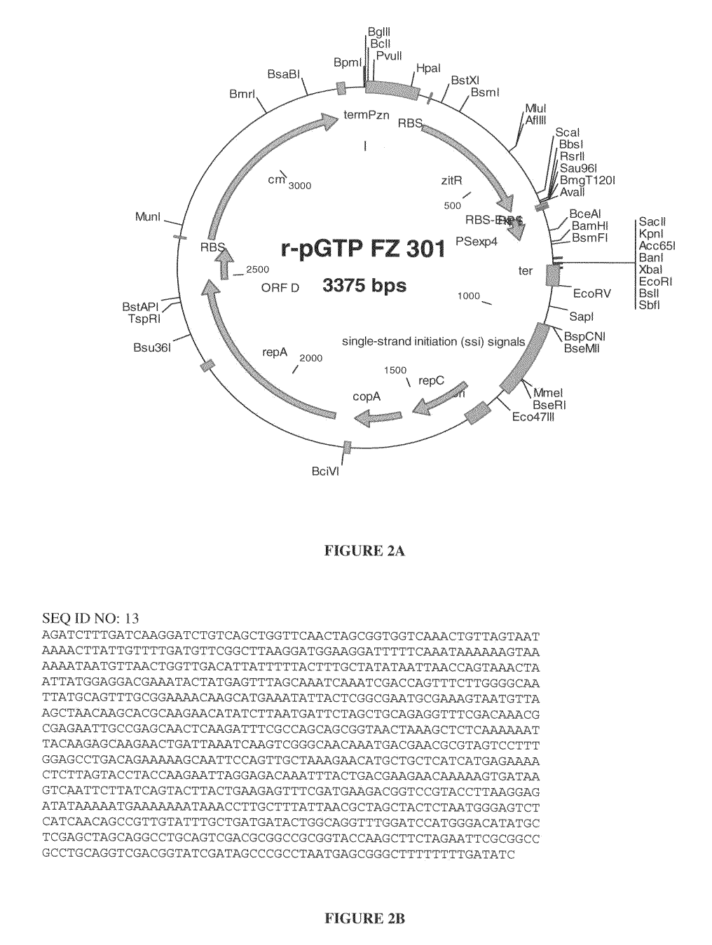

PGTP_FZ301 (FIG. 2A) is an expression vector for L. lactis derived from pLB145 (Morello et al., 2008) (FIG. 1). This vector permits expression of the gene of interest in translational fusion with the Exp4 signal peptide (Psexp4) dependent upon promoter P_{Zn} zitR (Pocquet et al., International Patent Application under the PCT published under no. WO 2004/020640). This vector was obtained through replacement of the PLB 145 expression cassette contained between the restriction sites BglII and EcoRV by a Z301 synthetic DNA sequence (FIG. 2B) including a multiple cloning site between the sequence coding for the Exp4 signal peptide and the transcription termination site.

### PGTP_FZR303

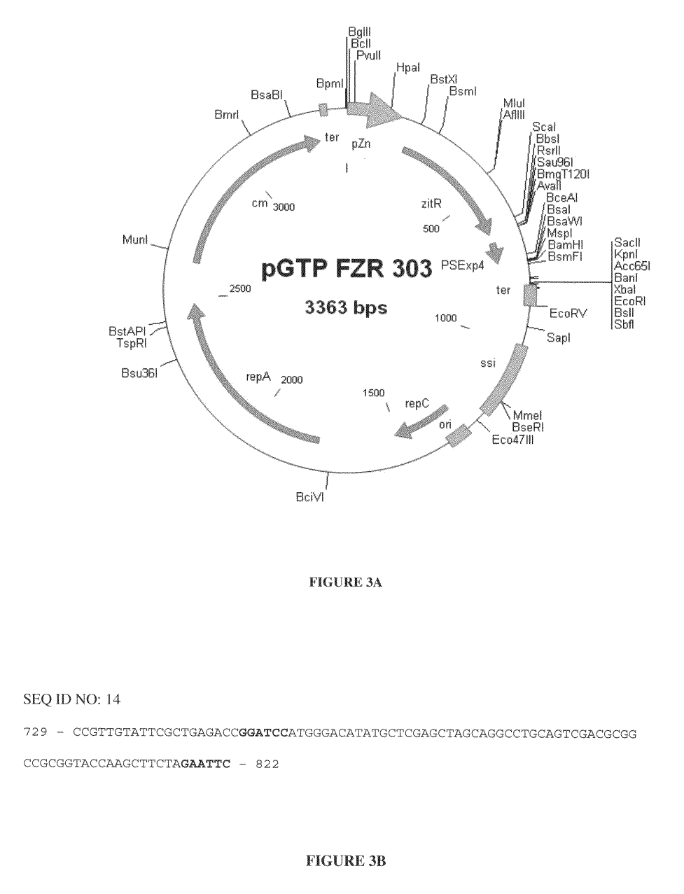

pGTP_FZR303 (FIG. 3A) is an expression vector derived from pGTP_FZ301. It was obtained through directed mutagenesis so as to insert a BsaI site downstream from the sequence coding for the EXP4 signal peptide and to match the BamHI site of this same sequence. The resulting sequence of this directed mutagenesis is indicated in FIG. 3B.

### APO Vector and NucB Vector

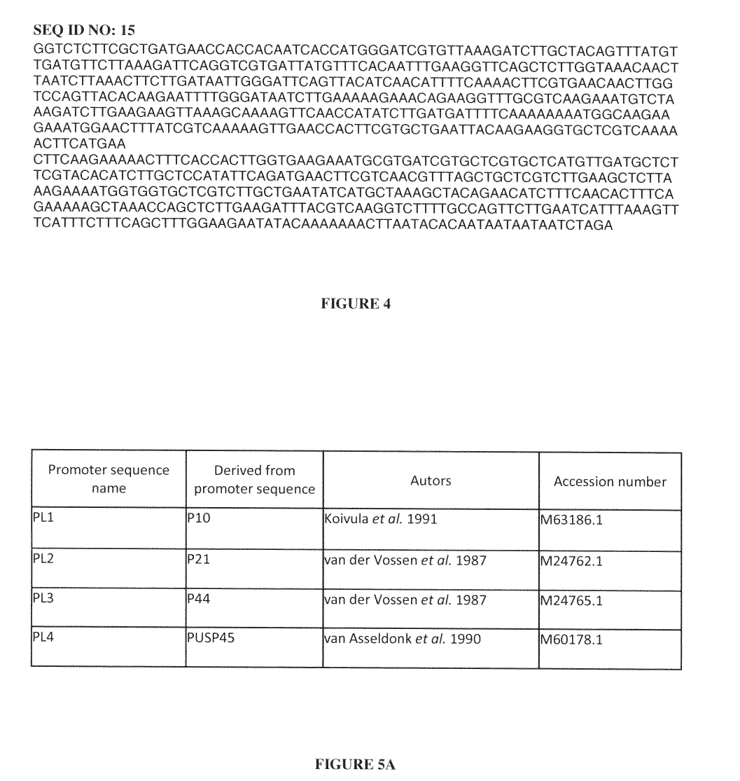

The nucleotide sequence coding for the human APO-AI human protein, modified to optimize the use of codons in L. lactis, was synthesized with the addition of restriction sites BsaI and XbaI (FIG. 4). This nucleotide sequence (SEQ ID NO: 15) was sub-cloned in L. lactis in the pGTP_FZR303 vector previously digested by BsaI and XbaI, yielding the construct pGTP_FZR303_{—}0600088.

The nucleotide sequence coding for the mature form protein NucB of Staphylococcus aureus (Davis et al., 1977) was amplified by PCR from the pLB145 vector (Morello et al., 2008) using the following primers:

(SEQ ID NO: 11)
5′-TTTAAATTTAGGATCCGCATCAAACAGATAACGG-3′
and
(SEQ ID NO: 12)
5′-TATATATATAGGTACCTTATTGACCTGAATCAGCGT-3′.

The PCR product obtained was then digested by the restriction enzymes BamHI and KpnI and subcloned in the previously digested pGTP_FZ301 vector. The resulting construction is called pGTP_FZ301_NucB.

### Promoters PL1, PL2, PL3 and PL4

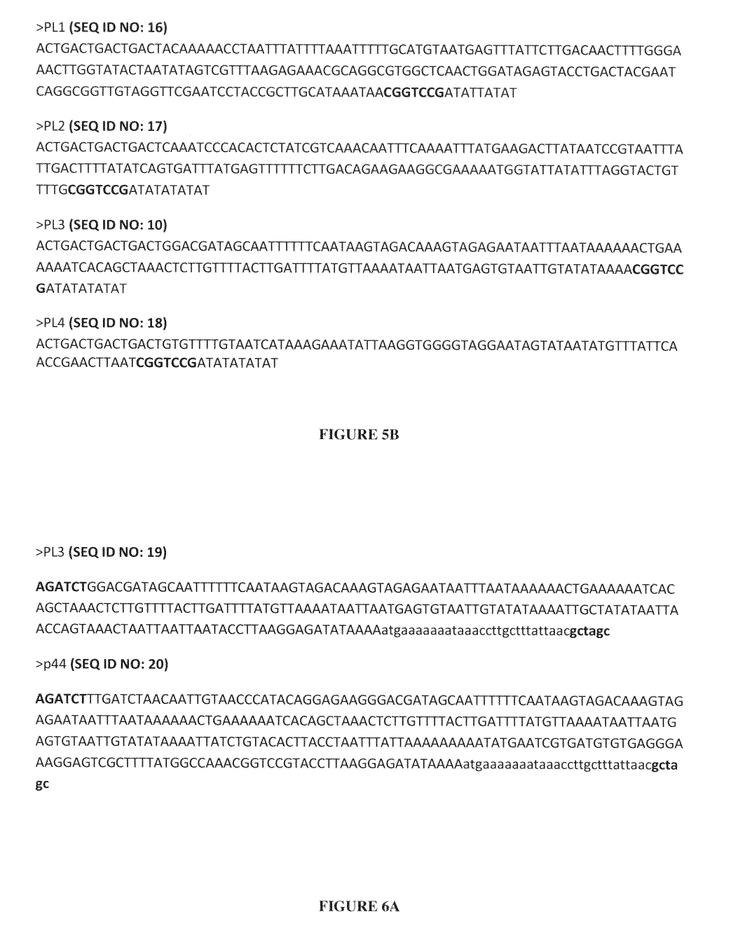

The nucleotide sequences (PL1, PL2, PL3 and PL4) correspond to truncated versions of constitutive promoter regions of L. lactis already described in the literature (Koivula et al., 1991; van der Vossen et al., 1987 and van Asseldonk et al., 1990, and FIG. 5A).

The various nucleotide sequences corresponding to the different promoters (PL1, PL2, PL3, PL4, PL5) (FIG. 5B) were synthesized and subcloned in the pGTP_FZR_{—}303_{—}0600088 vector previously digested by the enzymes PvuII and RsrII in L. lactis yielding the following vectors: pGTP_FPL1_{—}0600088, pGTP_FPL2_{—}0600088, pGTP_FPL2_{—}0600088 and pGTP_FPL4_{—}0600088.

### P44 and PL3

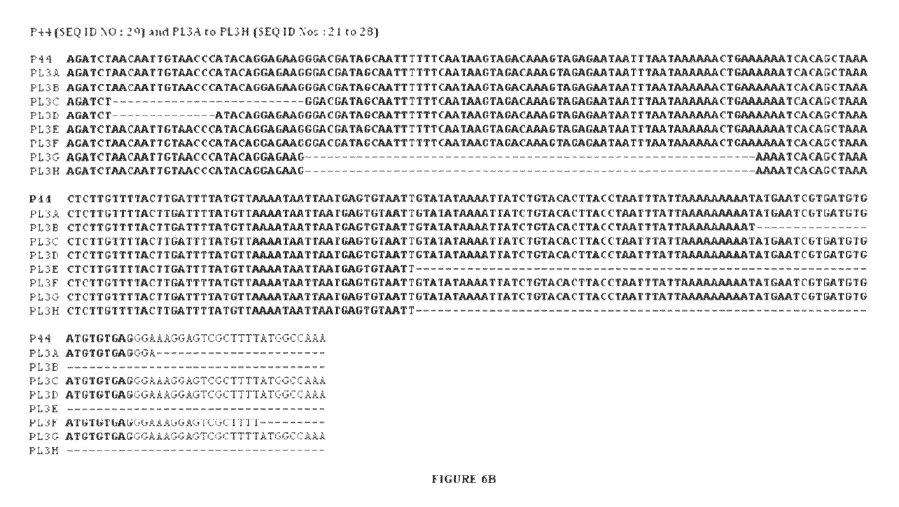

The nucleotide sequences corresponding to p44 (van der Vossen et al., 1987) and to the PL3 sequence followed by the 5′UTR region and nucleotides coding for the first amino acids of the EXP4 signal sequence were synthesized (FIG. 6A). The synthetic promoter sequence corresponding to p44 was digested by the restriction enzymes BglII and NheI and then subcloned in place of the PL3 promoter sequence in the expression vector pGTP_FLP3_{—}0600088 previously digested by BclI and NheI yielding the vector pGTP_Fp44_{—}0600088. The synthetic promoter sequences corresponding to p44 and the PL3 sequence were digested by the restriction enzymes BglII and NheI subcloned in the pGTP_F301 vector previously digested by BglII and NheI to yield the vectors pGTP_Fp44_NucB and pGTP_FPL3_NucB.

### Truncated PL3

The promoter sequences PL3A, PL3B, PL3C, PL3D, PL3E, PL3F, PL3G, PL3H (FIG. 6B) were synthesized. They were then digested by the restriction enzymes BgalII and NheI and subcloned in the pGTP_F301_NucB vector previously digested by BglII and NheI to yield respectively the following vectors:

pGTP_FPL3A_NucB, pGTP_FPL3C_NucB, pGTP_FPL3D_NucB, pGTP_FPL3E_NucB, pGTP_FPL3F_NucB, pGTP_FPL3G_NucB, pGTP_FPL3H_NucB.

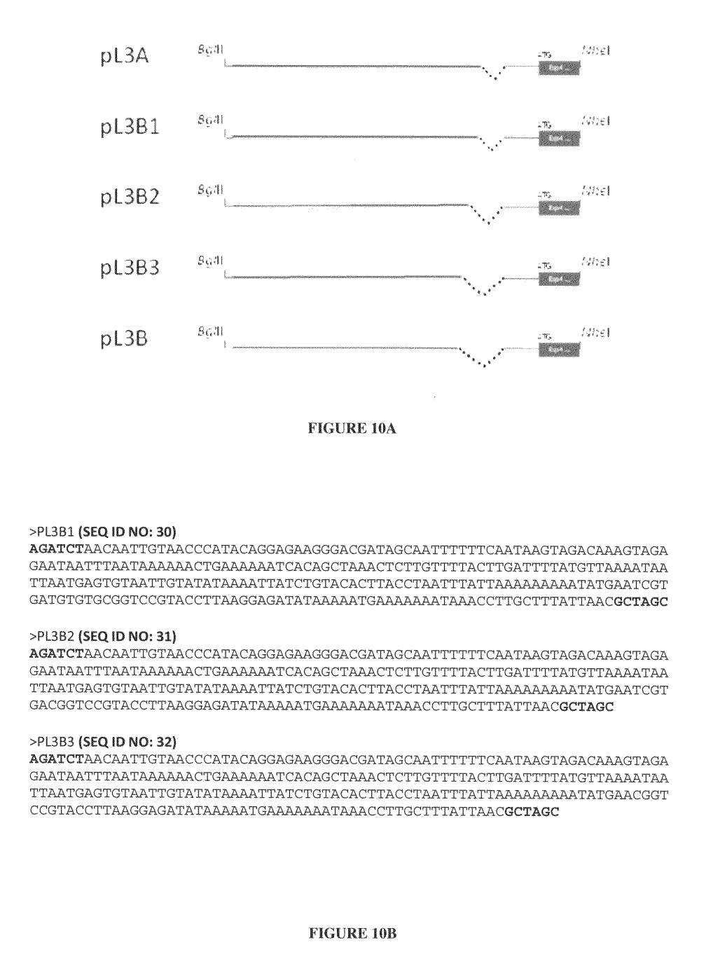

The promoter sequences PL3B1, PL3B2 and PL3B3 (FIGS. 10A-10B) were synthesized. They were then digested by the restriction enzymes BglII and NheI and subcloned in the pGTP_F301_NucB vector previously digested by BglII and NheI to yield respectively the following vectors:

pGTP_FPL3B1_NucB, pGTP_FPL3B2—NucB, pGTP_FPL3B3—NucB.

### Expression Vectors

| | Promoter | Protein | |
|---|---|---|---|
| Name | sequence | expressed | Parent plasmid |
| pGTP_FZ301 | P_{Zn}zitR | none | pLB145 |
| pGTP_FZR303 | P_{Zn}zitR | none | pGTP_FZ301 |
| pGTP_FZ301_NucB | P_{Zn}zitR | NucB | pGTP_FZ301 |
| pGTP_FZR303_0600088 | P_{Zn}zitR | ApoAI | pGTP_FZ303 |
| pGTP_FPL1_0600088 | PL1 | ApoAI | pGTP_FZ303_0600088 |
| pGTP_FPL2_0600088 | PL2 | ApoAI | pGTP_FZ303_0600088 |
| pGTP_FPL3_0600088 | PL3 | ApoAI | pGTP_FZ303_0600088 |
| pGTP_FPL4_0600088 | PL4 | ApoAI | pGTP_FZ303_0600088 |
| pGTP_Fp44_0600088 | p44 | ApoAI | pGTP_FPL3_0600088 |
| pGTP_Fp44_NucB | P44 | NucB | pGTP_FZ301_NucB |
| pGTP_FPL3_NucB | PL3 | NucB | pGTP_FZ301_NucB |
| pGTP_FPL3A_NucB | PL3A | NucB | pGTP_FZ301_NucB |
| pGTP_FPL3B_NucB | PL3B | NucB | pGTP_FZ301_NucB |
| pGTP_FPL3C_NucB | PL3C | NucB | pGTP_FZ301_NucB |
| pGTP_FPL3D_NucB | PL3D | NucB | pGTP_FZ301_NucB |
| pGTP_FPL3E_NucB | PL3E | NucB | pGTP_FZ301_NucB |
| pGTP_FPL3F_NucB | PL3F | NucB | pGTP_FZ301_NucB |
| pGTP_FPL3G_NucB | PL3G | NucB | pGTP_FZ301_NucB |
| pGTP_FPL3H_NucB | PL3H | NucB | pGTP_FZ301_NucB |
| pGTP_FPL3B1_NucB | PL3B1 | NucB | pGTP_FZ301_NucB |
| pGTP_FPL3B2_NucB | PL3B2 | NucB | pGTP_FZ301_NucB |
| pGTP_FPL3B3_NucB | PL3B3 | NucB | pGTP_FZ301_NucB |

### Bacterial Strains and Culture Conditions

The intermediate subcloning operations were performed in Escherichia coli NEB 5-a (New England Biolabs, Ipswich, Mass.) in PUC-type subcloning vectors. Escherichia coli was cultivated in Luria Bertani (LB) medium: 1% tryptone (Sigma, St Louis, Mo.), 5% yeast extract (Fluka, St Louis Mo.), 1% NaCl (Fluka) supplemented with ampicillin at 100 μg/mL (Sigma Aldrich) at 37° C. on a shaker platform with agitation of 200-250. The solid medium was prepared by the addition of agar (Invitrogen, Paisley, UK) at a concentration of 1.5% weight/volume.

The subcloning phases in the expression vectors were performed in the MG1363 strain of Lactococcus lactis MG 1363 (Gasson et al., 1983) cultivated at 30° C. without agitation in a rich M17 medium (Fluka) supplemented with 1% glucose and 5 μg/mL chloramphenicol (Sigma Aldrich) for the plasmid selection.

For the expression phases of the heterologous proteins, the recombinant strains of Lactococcus lactis MG1363 or LA17 (MG1363 htrA—GTP Technology) derived from a pre-culture of 14 hours in GM17v medium were inoculated at an optical density of 600 nm (D0600) of 0.2 in 200 mL mini-bioreactors and cultivated under slow agitation (100 to 500 rpm) at 30° C. in GM17v medium without aeration and at a pH regulated to 6.5 by the addition of 30% NH₄OH (Fluka). The GM17v medium contains 1% vegetable extracts (Fluka), 0.25% yeast extract, 0.05% L-ascorbic acid (Sigma), 1.9% sodium gylcerophosphate (Sigma), 0.05% MgSO₄ (Fluka) and 5% glucose. The bacterial growth of these cultures was followed every hour by measurement of the optical density at 600 nm.

### Analysis of Expression

In order to evaluate the expression of each construct, aliquots of culture supernatants were sampled and placed SDS PAGE gel stained with Coomassie blue. The intensity of each band corresponding to the protein of interest was analyzed using densitometry (BIORAD GS800 and ImageQuantTL, Amersham Bioscience). The quantity of each protein present in the supernatant was determined relative to a protein reference range (BSA) placed on the same gel. In the case of the Apo-AI protein, since the initial expression level did not allow direct quantification on SDS PAGE gel, relative quantification of the expression was performed with densitometric analysis of the intensity of the band revealed in a Western blot with an anti Apo-AI antibody (AutogenBioclear #RP-063).

### EXAMPLE 2

### Comparison of the Efficacy of Promoter Sequences PL1, PL2, PL3 and PL4 for Expression of APO-AI

Four DNA sequences corresponding to promoter sequences of L. lactis were compared for their capacity to permit expression of the Apo-AI protein in the culture supernatant. Comparative analysis of the expression of the APO-AI recombinant protein with these 4 promoter sequences PL1, PL2, PL3 and PL4 was carried out as follows.

The expression vectors pGTP_FPL1_{—}0600088, pGTP_FPL2_{—}0600088, pGTP_FPL3_{—}0600088 and pGTP_FPL4_{—}0600088 were transformed in the LA17 (L. lactis) strain. A transforming clone was chosen for each construct and cultivated in GM17v medium at 30° C. without aeration and a regulated pH of 6.5 with the addition of 30% NH₄OH. Kinetic samples were taken every hour after 3 hours of culturing and analyzed with a Western blot in order to determine the relative quantity of APO-AI present in each sample.

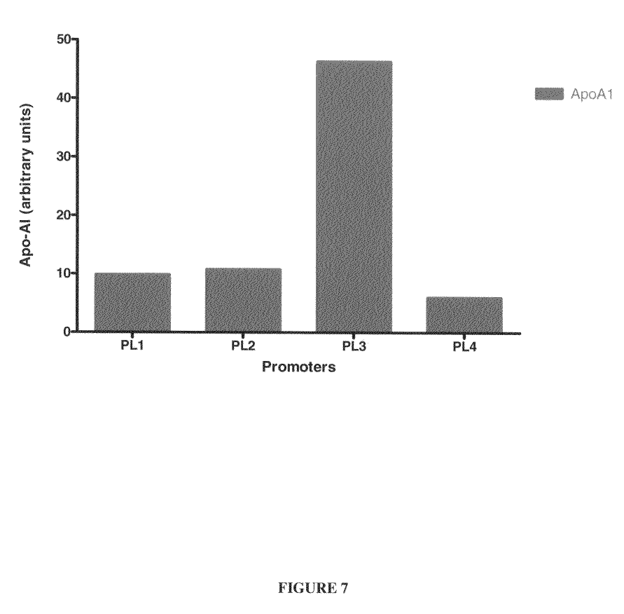

No significant different in bacterial growth was noted for the 4 strains evaluated. Analysis of the comparative expression of the protein of interest in the culture supernatant reveals that whatever promoter sequence was evaluated, the quantity of Apo-AI protein increases as a function of the culture time, reaching a maximum after 8 hours of culturing. Analysis of the relative quantity of Apo-AI present in the culture supernatant after 8 hours is presented in FIG. 7. It reveals that use of the PL3 promoter sequence permits an expression 4 to 5 times greater to be obtained than that obtained with the other promoter sequences (PL1, PL2 and PL4). The PL3 and PL2 sequences correspond to truncated forms of promoters p44 and p21 described in van der Vossen et al., 1987. In this publication, the authors use a comparative analysis of the transcriptional activity of these promoters to reveal that the level of expression obtained with the p44 sequence is 6 times higher than that obtained with the p21 sequence. However, the results obtained in the expression trials presented in FIG. 7 reveal the opposite result. In fact, the promoter derived from p44 (PL3) permits a significantly higher expression than that obtained with the promoter derived from p21 (PL2). These results suggest that the deletions performed in these promoter sequences modify the transcriptional activity of these promoters and lead to an improvement in the expression obtained from p44 and/or a decrease in the expression obtained from P21.

### EXAMPLE 3

### Comparison of the Efficacy of PL3 and P44 Promoter Sequences for the Expression of APO-AI

In order to evaluate the importance of the improvement of the promoter obtained through deletion of the sequences upstream and downstream of the p44 sequence, a comparative analysis of the expression of these two proteins under the dependence of promoters p44 and PL3 was performed.

The expression vectors pGTP_FPL3_{—}0600088 and pGTP_FP44_{—}0600088 were transformed. in the LA17 strain (L. lactis). The expression vectors pGTP_FP44_NucB and pGTP_FPLE_NucB were transformed in the MG1363 strain (L. lactis). A transforming clone was chosen for each of the constructs and cultivated in GM17v medium at 30° C. without aeration and at a regulated pH of 6.5 with the addition of 30% NH₄OH. Kinetic samples were taken every hour after 3 hours of culturing and analyzed in a Western blot in the case of the expression of the Apo-AI protein and with densitometric analysis on SDS-PAGE gel colored with Coomassie blue in the case of the NucB protein.

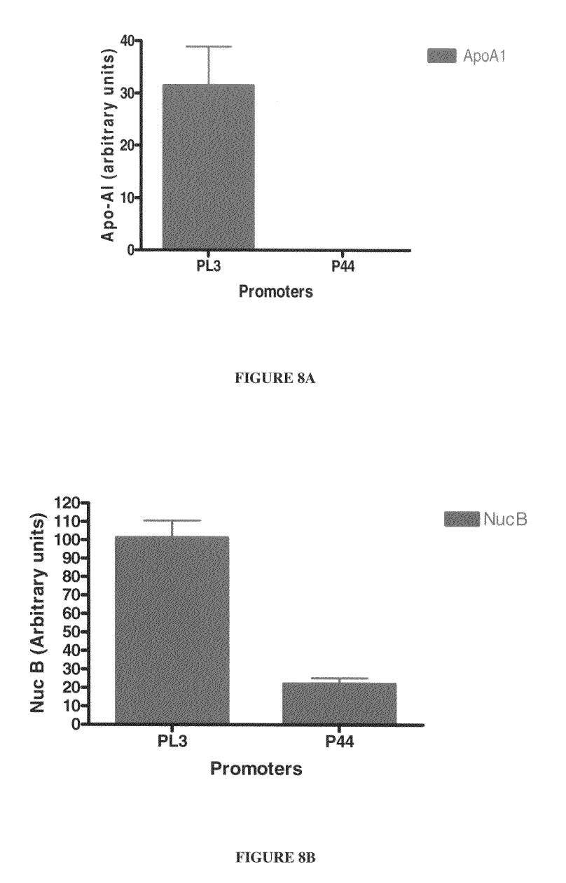

In accordance with what was noted during comparison of the PL1, PL2, PL3 and Pl4 promoters, a maximal accumulation of each of the proteins of interest in the culture supernatant after 8 hours of culturing was noted. Comparative analysis of the expression of two proteins NucB and Apo-AI after 8 hours of culturing is presented in FIG. 8. We note that the expression of the Apo-AI protein is significantly greater with the PL3 promoter. In fact, no expression of this protein could be detected with the p44 promoter, whereas this protein is well-expressed under the dependence of the PL3 promoter (FIG. 8A). In addition, it should be noted that—in agreement with the results previously published (van der Vossen et al., 1987)—we note that the expression provoked by promoter p44 is significantly lower than that obtained with the PL2 promoter sequence derived from p21. Concerning the protein NucB (FIG. 8B), we note that expression of NucB with the promoter PL3 is 5 times greater than that obtained with the p44 promoter. Therefore, these analyses of 2 different proteins clearly demonstrate the positive effect of deletions performed in the p44 sequence. Surprisingly, these deletions enable a significant increase in the level of expression in the L. lactis supernatant. Thus, use of the truncated promoter PL3 enables increasing the level of expression of recombinant proteins in the culture supernatant by at least a factor of 5.

### EXAMPLE 4

### Definition of the Optimal Promoter Sequence

In order to characterize more precisely the terminals of the optimal promoter sequence, several other deletions of the p44 sequence were performed (FIGS. 6A-6B). A comparative analysis of the expression of the NucB protein under the dependence of these different proteins was performed.

The expression vectors pGTP_FP44—NucB, pGTP_FPL3_NucB, pGTP_FPL3A_NucB, pGTP_FPL3B_NucB, pGTP_FPL3C_NucB, pGTP_FPL3D_NucB, pGTP_FPL3E_NucB, pGTP_FPL3F_NucB, pGTP_FPL3G_NucB and pGTP_FPL3H_NucB, were transformed in the MG1363 strain (L. lactis). A transforming clone was chosen for each of the constructs and cultivated in GM17v medium at 30° C. without aeration and at a regulated pH of 6.5 with the addition of 30% NH₄OH. Kinetic samples were taken every hour after 3 hours of culturing and analyzed with densitometric analysis on SDS-PAGE gel stained with Coomassie blue.

### Results

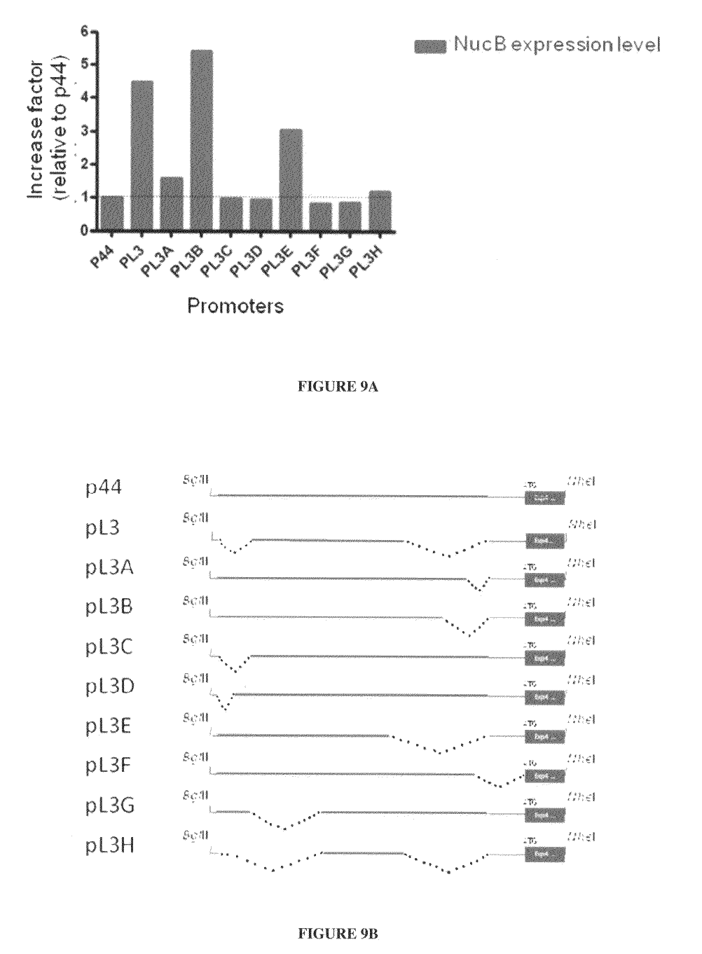

### See FIGS. 9A and 9B

### EXAMPLE 5

### Definition of the Downstream Terminal of the Optimal Promoter

In order to better define the optimal terminus of the promoter sequence, three new deletions were performed between promoter PL3A and PL3B. The three promoter sequences PL3B1, PL3B2 and PL3B3 have been synthesized (FIGS. 10A and 10B) and a comparative analysis of the expression of the NucB protein under the dependence of these different promoters was performed.

The expression vectors, pGTP_FPL3B1_NucB, pGTP_FPL3B2_NucB, pGTP_FPL3B3_NucB, pGTP_FPL3B_NucB and pGTP_FP44_NucB, were transformed in the MG1363 strain (L. lactis). A transforming clone was chosen for each of the constructs and cultivated in GM17v medium at 30° C. without aeration and at a regulated pH of 6.5 with the addition of 30% NH₄OH. Kinetic samples were taken every hour after 3 hours of culturing and analyzed with densitometric analysis on SDS-PAGE gel stained with Coomassie blue.

### Results

Interestingly, it appeared that NucB expression in the culture supernatant is similar and optimal when the protein of interest is expressed under the control of promoters PL3, PL3B and pL3B3. On the other hand, the protein expression driven under the control of promoters PL3B2 and PL3B1 is similar to what is obtained with PL3A which only represents 20 to 25% of the optimal expression yield.

### REFERENCES

Koivula, T., Sibakov, M. and Palva, I.: Isolation and characterization of Lactococcus lactis subsp. Lactis promoters. Appl. Environ. Microbiol. 57 (2), 333-340 (1991).
Van der Vossen, J. M., van der Lelie, D. and Venema, G: Isolation and characterization of Streptococcus cremoris Wg2-specific promoters. Appl. Environ. Microbiol. 53 (10), 2452-2457 (1987).
Van Asseldonk, M., Rutten, G., Oteman, M., Seizen, R. J., de Vos, W., Mand Simons, G.: Cloning of usp45, a gene encoding a secreted protein Lactococcus lactis subsp. Lactis MG1363. Gene 95 (1), 155-160 (1990).
Gasson, M. J.: Plasmid complements of Streptococcus lactis NCDO 712 and other lactic streptococci after protoplast-inducing curing. J. Bacteriol. 1983, 154(1): 1-9.
Morello, E., Bermudez-Humaran, L. G., Llull, D., Sole, V., Miraglio, N., Langella, P., Poquet, I.: Lactococcus lactis, an efficient cell factory for recombinant protein production and secretion. J. Mol. Microbiol. Biotechnol. 2008, 14 (103):48-58.
Davis, A., Moore I B, Parker D S, Taniuchi H: Nuclease B. A possible precursor of nuclease A, an extracellular nuclease of Staphylococcus aureus. J Biol Chem 1977, 252 (18): 6544-6553.
Hyyryläinen H L, Sarvas M, Kontinen V P: Transcriptome analysis of the secretion stress response of Bacillus subtilis. Appl Microbiol Biotechnol. 2005 May; 67(3): 389-96. Epub 2005 Jan. 27.
De Vos W M, Beerthuyzen M M, Luesink E L, Kuipers O P.: Genetics of the nisin operon and the sucrose-nisin conjugative transposon Tn5276. Dev Biol Stand. 1995; 85:617-25.
Kuipers O P, Rollema H S, Siezne R J, De Vos W M: Lactococcal expression systems for protein engineering of nisin. Dev Biol Stand. 1995; 85:605-13.
Miyoshi A, Jamet E, Commissaire J, Renault P, Langella P, Azevedo V.: A xylose-inducible expression system for Lactococcus lactis. FEMS Mircobiol Lett. 2004 Oct. 15; 239 (2): 205-12.
Madsen S M, Arnau J, Vrang A, Givskov M, Israelsen H: Molecular characterization of the pH-inducible and growth phase-dependent promoter P170 of Lactococcus lactis. Mol. Microbiol. 1999 April; 32 (1):75-87.
Llull D, Poquet I.: New expression system tightly controlled by zinc availability in Lactococcus lactis. Appl Environ Microbiol. 2004 September; 70 (9):5398-406.
Nauta A, van den Burg B, Karsens H, Venema G, Kok J.: Design of thermolabile bacteriophage repressor mutants by comparative molecular modeling. Nat. Biotechnol. 1997 October; 15 (10):980-3.
Walker, S. A., Klaenhammer, T. R.: Molecular characterization of a phage-inducible middle promoter and its transcriptional activator from the lactococcal bacteriophage phi31. J. Bacteriol. 1998 February; 180 (4):921-31.
De Vos, W. M.: Gene expression systems for lactic acid bacteria. Curr Opin Microbiol. 1999 June; 2(3):289-95.

1. Isolated nucleic acid characterized in that it is:
a) a fragment of the SEQ ID NO: 1 sequence, said fragment being characterized in that it is chosen from the fragments comprised in the nt 1-217, terminals included, of the SEQ ID NO: 1 sequence and containing at least the nt 88-149 sequence (SEQ ID NO: 3) of the SEQ ID NO: 1 sequence; or
b) a fragment whose sequence is at least 90% identical to the sequence of the fragment as defined in a).

2. Isolated nucleic acid according to claim 1, characterized in that it is:
a) a fragment of the SEQ ID NO: 1 sequence, said fragment being characterized in that it is chosen from the fragments comprised in the nt 1-195 sequence (SEQ ID NO: 2), terminals included, of the SEQ ID NO: 1 sequence and containing at least the nt 88-149 sequence (SEQ ID NO: 3) of the SEQ ID NO: 1 sequence; or
b) a fragment whose sequence is least 90% identical to the sequence of the fragment as defined in a).

3. Nucleic acid according to claim 1, characterized in that said fragment is chosen from:
a) a fragment comprised in the nt 1-195 sequence (SEQ ID NO: 2), terminals included, of the SEQ ID NO: 1 sequence and containing at least the nt 88-159 sequence (SEQ ID NO: 4) of the SEQ ID NO: 1 sequence; or
b) a fragment whose sequence is at least 90% identical to the sequence of the fragment as defined in a).

4. Nucleic acid according to claim 1, characterized in that said fragment is chosen from:
a) a fragment contained in the nt 1-195 sequence (SEQ ID NO: 2), terminals included, of the
SEQ ID NO: 1 sequence and containing at least the nt 27-149 sequence (SEQ ID NO: 5) of the SEQ ID NO: 1 sequence; or
b) a fragment whose sequence is at least 90% identical to the sequence of the fragment as defined in a).

5. Nucleic acid according to claim 1, characterized in that said fragment is chosen from:
a) fragment contained in the nt 1-195 sequence (SEQ ID NO: 2), terminals included, of the SEQ ID NO: 1 sequence and containing at least the nt 27-159 sequence (SEQ ID NO: 6) of the SEQ ID NO: 1 sequence; or
b) a fragment whose sequence is at least 90% identical to the sequence of the fragment as defined in a).

6. Nucleic acid isolated according to claim 1, characterized in that in a) said fragment is contained in the nt 15-195 sequence (SEQ ID NO: 7), terminals included, of the SEQ ID NO: 1 sequence.

7. Nucleic acid isolated according to claim 1, characterized in that in a), said fragment is contained in the nt 27-195 sequence (SEQ ID NO: 8), terminals included, of the SEQ ID NO: 1 sequence.

8. Nucleic acid isolated according to claim 1, characterized in that in a), said fragment is contained in the nt 15-159 sequence (SEQ ID NO: 9), terminals included, of the SEQ ID NO: 1 sequence.

9. Nucleic acid according to claim 1, characterized in that said fragment is chosen from the fragments of SEQ ID NO: 2 to SEQ ID NO: 9 sequences or a fragment whose sequence is at least 90% identical to one of the SEQ ID NO: 2 to SEQ ID NO: 9 sequences.

10. Nucleic acid according to claim 1, characterized in that said fragment is chosen from:
a) the fragment having the sequence nt 27459 (SEQ ID NO: 6); or
b) a fragment whose sequence is at least 90% identical to the sequence of the fragment as defined in a).

11. Nucleic acid according to claim 1 with promoter activity.

12. Promoter comprising a nucleic acid according to claim 1 or containing as a promoter sequence a nucleic acid according to claim 1.

13. Expression vector, characterized in that it contains as promoter a nucleic acid according to claim 11.

14. Expression vector, characterized in that it contains as promoter a promoter according to claim 12.

15. Expression vector carrying a gene of interest, characterized in that said gene of interest is under the control of a nucleic acid according to claim 11.

16. Expression vector carrying a gene of interest, characterized in that said gene of interest is under the control of a promoter according to claim 12.

17. Expression vector according to claim 15, characterized in that it also includes the means necessary for the expression of said gene of interest, its replication and, if appropriate, the selection of transformed cells.

18. Expression vector according to claim 16, characterized in that it also includes the means necessary for the expression of said gene of interest, its replication and, if appropriate, the selection of transformed cells.

19. Expression vector according to claim 13, characterized in that the vector carrying the nucleic acid according to claim 11 is a vector chosen from the pGTP_FZ301 vector, the pGTP_FZR303 vector, or any expression vector adapted to the expression of a heterologous protein in Lactococcus lactis.

20. Expression vector according to claim 14, characterized in that the vector carrying the promoter according to claim 12 is a vector chosen from the pGTP_FZ301 vector, the pGTP_FZR303 vector, or any expression vector adapted to the expression of a heterologous protein in Lactococcus lactis.

21. Expression vector according to claim 15, characterized in that the gene of interest is a recombinant gene coding for a therapeutic, vaccine-related or immunogenic, diagnostic, cosmetic or agri-food protein.

22. Expression vector according to claim 16, characterized in that the gene of interest is a recombinant gene coding for a therapeutic, vaccine-related or immunogenic, diagnostic, cosmetic or agri-food protein.

23. Host cell, characterized in that it is transformed by an expression vector according to claim 13.

24. Host cell, characterized in that it is transformed by an expression vector according to claim 14.

25. Host cell transformed according to claim 23, characterized in that it is a bacterium, preferably of genus Lactococcus, in particular Lactococcus lactis.

26. Host cell transformed according to claim 24, characterized in that it is a bacterium, preferably of genus Lactococcus, in particular Lactococcus lactis.

27. Method for the production of a heterologous protein containing the culture of a host cell according to claim 23.

28. Method for the production of a heterologous protein containing the culture of a host cell according to claim 24.

29. Method for the production of a heterologous protein in a cell, characterized in that it contains:
a) the culture of a host cell according to claim 23 transformed by a vector or nucleic acid containing a gene of interest coding for said heterologous protein, under conditions permitting the stable expression of said protein; and
b) the extraction of the heterologous protein from cells or culture supernatant.

30. Method for the production of a heterologous protein in a cell, characterized in that it contains:
a) the culture of a host cell according to claim 24 transformed by a vector or nucleic acid containing a gene of interest coding for said heterologous protein, under conditions permitting the stable expression of said protein; and
b) the extraction of the heterologous protein from cells or culture supernatant.

31. Method for the production of a heterologous protein in a cell according to claim 29, characterized in that the heterologous protein is a secreted protein.

32. Method for the production of a heterologous protein in a cell according to claim 30, characterized in that the heterologous protein is a secreted protein.
